(19) United States
(12) Patent Application Publication
(10) Pub. No.: US 2021/0393822 A1
(43) Pub. Date: Dec. 23, 2021

(54) VEHICLE CABIN UV LAMP SANITIZER

(71) Applicant: RAB Lighting Inc., Northvale, NJ (US)

(72) Inventors: Benjamin Chen, Northvale, NJ (US); Joseph Ernest Minns, Monroe, NY (US); Shi Shi, Shanghai (CN); Xu Yunfei, Shanghai (CN); Wen Tianxiang, Shanghai (CN); Ding Xianlong, Shanghai (CN); Bi Yongsi, Shanghai (CN)

(21) Appl. No.: 17/350,589

(22) Filed: Jun. 17, 2021

Related U.S. Application Data
(60) Provisional application No. 63/041,608, filed on Jun. 19, 2020.
(60) Provisional application No. 63/040,028, filed on Jun. 17, 2020.

(51) Int. Cl. A61L 2/10; B60S 3/00

(52) U.S. Cl. CPC A61L 2/10; B60S 3/008; A61L 2209/15; A61L 2202/16; A61L 2202/25; A61L 2202/11

(57) ABSTRACT

A sanitizing UV lamp device for disinfecting surfaces in spaces used by people, including a motor vehicle cabin, includes a housing having a cup shaped base, UV lamps powered for a selected period of time by the control circuit, a reflector, and a protective screen surrounding the reflector and the UV lamps to provide 360-degree germicidal irradiation through openings defined by the screen. The UV lamp device also includes various safety features, including occupancy detection to prevent irradiation from the at least one UV lamp upon detection of motion.

### CROSS-REFERENCE TO RELATED APPLICATIONS

This application claims the benefit of U.S. Provisional Application No. 63/040,028, filed Jun. 17, 2020, and U.S. Provisional Application No. 63/041,608, filed Jun. 19, 2020, which are each hereby incorporated herein by reference.

### TECHNICAL FIELD

Embodiments of this disclosure relate generally to germicidal irradiation devices for surfaces, and more particularly to UV lamp sanitizing devices.

### BACKGROUND

Businesses that service motor vehicles have experienced an increase in demand for and owners of vehicles have shown increased interest in disinfecting the interior/cabin of motor vehicles. Typical methods use chemical disinfectants that can cause surface deterioration, trigger chemical sensitivities for some people, are labor intensive, and use expensive consumables.

As such, it was realized by the inventors of the current disclosure that improvements in devices and methods to sanitize motor vehicle cabins are needed.

### SUMMARY

Ultraviolet (UV) germicidal irradiation uses short-wavelength ultraviolet light produced by a UV lamps to kill microorganisms such as viruses, bacteria, mold, and other pathogens. UV lamps, for example, ultraviolet C or UV-C lamps, have been used to effectively disinfect water and air streams, and to sanitize food products.

The present disclosure is directed to embodiments of a portable sanitizing UV lamp device that provides many advantages for sanitizing motor vehicle cabins or, alternatively or additionally, other interior spaces people use and for which non-contact, non-chemical sanitization is desirable.

The exemplary sanitizing UV lamp device provides disinfection of surfaces in spaces used by people, including a motor vehicle cabin, and includes a housing having a cup shaped base, germicidal UV lamps powered for a selected period of time by the control circuit, a reflector, and a protective screen surrounding the reflector and the UV lamps to provide 360-degree germicidal irradiation through openings defined by the screen. The UV lamp device also includes various safety and other advantageous features, including occupancy detection to prevent irradiation from the at least one UV lamp upon detection of motion, audible warning during a brief delay prior to initiating irradiation, wireless control, and lifespan monitoring of replaceable UV lamps.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein does not necessarily address every object described herein, and each embodiment does not necessarily include each feature described. Other forms, embodiments, objects, advantages, benefits, features, and aspects of the present disclosure will become apparent to one of skill in the art from the detailed description and drawings contained herein. Moreover, the various apparatuses and methods described in this summary section, as well as elsewhere in this application, can be expressed as a large number of different combinations and sub-combinations. All such useful, novel, and inventive combinations and sub-combinations are contemplated herein, it being recognized that the explicit expression of each of these combinations is unnecessary.

### BRIEF DESCRIPTION OF THE DRAWINGS

Some of the figures shown herein may include dimensions or may have been created from scaled drawings. However, such dimensions, or the relative scaling within a figure, are by way of example, and not to be construed as limiting.

FIG. 1 is a perspective assembly view of an exemplary embodiment of a sanitizing UV lamp device according to the present disclosure;

FIG. 2 is a planform view illustrating an exemplary remote control for use with the UV lamp device of FIG. 1;

FIG. 3 is a perspective view of the sanitizing UV lamp device of FIG. 1 prepared for use in a motor vehicle cabin;

FIG. 4 is a partially exploded view of the sanitizing UV lamp device of FIG. 1;

FIG. 5 is a horizontal cross-sectional view taking along cutline 5-5 FIG. 1 and illustrates the UV lamp bulb, reflector, and cage configuration of the sanitizing UV lamp device and FIG. 1;

FIG. 6 is a top perspective view of the sanitizing UV lamp device of FIG. 1;

FIG. 7 is a bottom perspective view of an upper lamp portion of the sanitizing UV lamp device of FIG. 1 and illustrates replacement of a UV lamp bulb;

FIG. 8 is a horizontal cross-sectional view taken along cutline 8-8 of FIG. 5 and illustrates the locking mechanism securing the upper lamp portion of the sanitizing UV lamp device of FIG. 1;

FIG. 9 is a horizontal cross-sectional view taken along cutline 9-9 of FIG. 5 and illustrates the telescoping height adjust release mechanism of the lower support portion of the sanitizing UV lamp device of FIG. 1;

FIG. 10 is an exemplary electrical schematic block diagram of the sanitizing UV lamp device of FIG. 1; and

FIG. 11 is a flow chart illustrating an exemplary algorithm associated with the electrical schematic block diagram of FIG. 10.

### DETAILED DESCRIPTION OF THE ILLUSTRATED EMBODIMENTS

For the purposes of promoting an understanding of the principles of the disclosure, reference will now be made to one or more embodiments, which may or may not be illustrated in the drawings, and specific language will be used to describe the same. It will nevertheless be understood that no limitation of the scope of the disclosure is thereby intended; any alterations and further modifications of the described or illustrated embodiments, and any further applications of the principles of the disclosure as illustrated herein are contemplated as would normally occur to one skilled in the art to which the disclosure relates. At least one embodiment of the disclosure is shown in great detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to benefits or advantages provided by some embodiments, other embodiments may not include those same benefits or advantages, or may include different benefits or advantages. Any benefits or advantages described herein are not to be construed as limiting to any of the claims.

Likewise, there may be discussion with regards to “objects” associated with some embodiments of the present invention, it is understood that yet other embodiments may not be associated with those same objects, or may include yet different objects. Any advantages, objects, or similar words used herein are not to be construed as limiting to any of the claims. The usage of words indicating preference, such as “preferably,” refers to features and aspects that are present in at least one embodiment, but which are optional for some embodiments.

Specific quantities (spatial dimensions, temperatures, pressures, times, force, resistance, current, voltage, concentrations, wavelengths, frequencies, heat transfer coefficients, dimensionless parameters, etc.) may be used explicitly or implicitly herein, such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated.

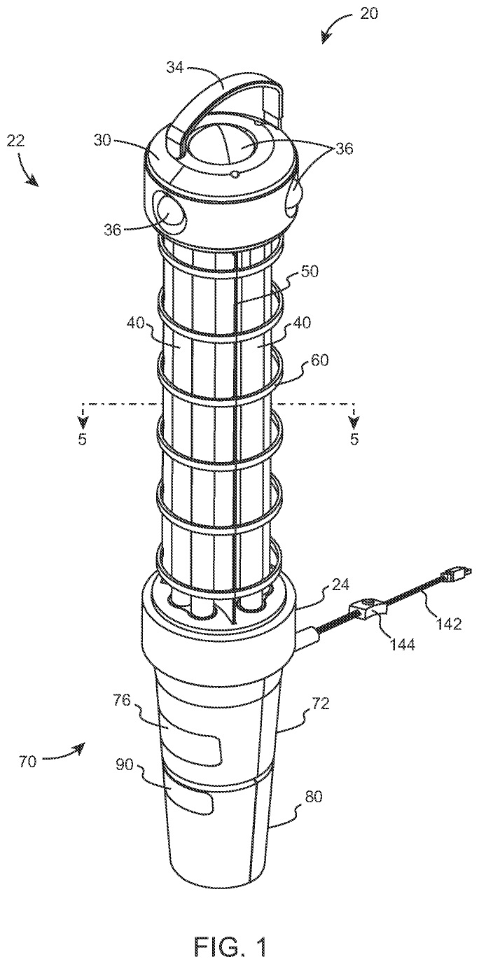

Referring to FIG. 1, an exemplary embodiment of a sanitizing UV lamp device 20 according to the present disclosure is illustrated. The device 20 includes generally a lamp support portion 22 and a lower base support portion 70. The lamp support portion 22 and lower base support portion 70 includes a housing generally constructed of polycarbonate or another rigid material. The lamp support portion 22 of the housing includes a lower housing 24 and an upper housing 30. The lower base support portion 70 of the housing includes a coupling housing 72 and a cup base housing 80.

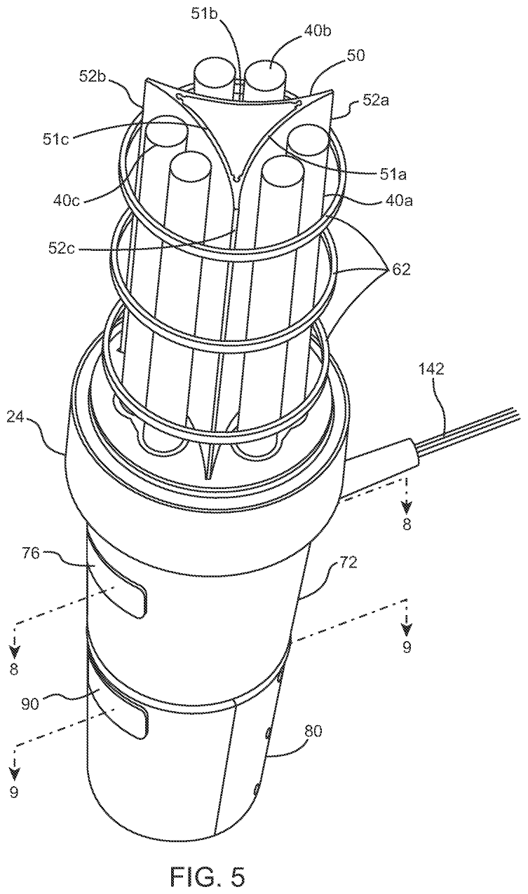

The lamp support portion 22 includes at least one germicidal UV lamp 40 supported between the lower housing 24 and the upper housing 30, and may also include a reflector 50, and a safety screen 60 surrounding the UV lamps and reflector. For example, as best shown in FIG. 5, an exemplary embodiment of the device 20 includes three UV lamps 40a-40c located around a central reflector 50. The reflector 50 is formed by three reflector walls 51a-51c joined together at apexes 52a-52c, forming a closed generally triangular cross-sectional structure. This arrangement advantageously provides germicidal irradiation through a 360-degree radius around the device 20. UV lamps 40 may be, for example, widely available 36 watt, ozone free quartz UV-C H type twin tube sterilization lamp, also known as model APL-L36W, and having a 2G11 lamp base.

Advantageously, the protective lamp screen 60 can be formed, for example, by cage rings 62 that are periodically coupled to the apexes 52 of and along the length of reflector 50. Lamp screen 60 provides protection against inadvertent breakage of the UV lamps 40 and a burn by inadvertent touching of the lamps. A further safety feature providing the same advantages as the screen 60 is a handle 34 coupled to the upper housing 30 that can be used to grasp and move the device 20. Reflector 50, screen 60, and handle 34 can be formed from 6030 aluminum alloy or other rigid materials known in the art.

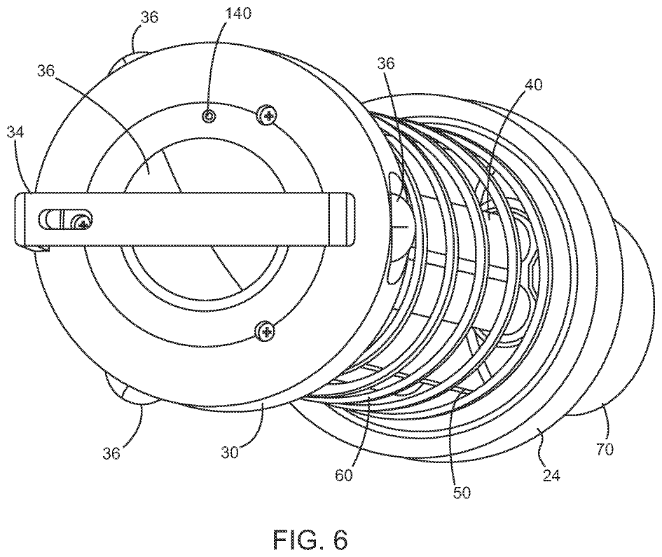

Because the emitted light wavelength used by UV lamps 40 for germicidal irradiation presents a risk to people and animals, another safety feature of the device 20 is occupancy and/or vacancy detection. For example, PIR sensors 132 located under sensor covers 36, for example three located uniformly around the periphery of and one located on top of the upper housing 30 in the exemplary embodiment, as shown in FIGS. 1 and 6, thereby providing detection within the entire space to be irradiated, including for example, detection of any door or window associated with the motor vehicle interior 12 being opened.

A power supply cord 142 providing electrical power to an electrical circuit 120 of the device 20, for example 120 volts AC or an alternative standard electrical supply, can include a safety shutoff switch 144, for example, a normally closed switch that may be manually actuated to quickly interrupt the power supplied to the device 20, including the UV lamps 40.

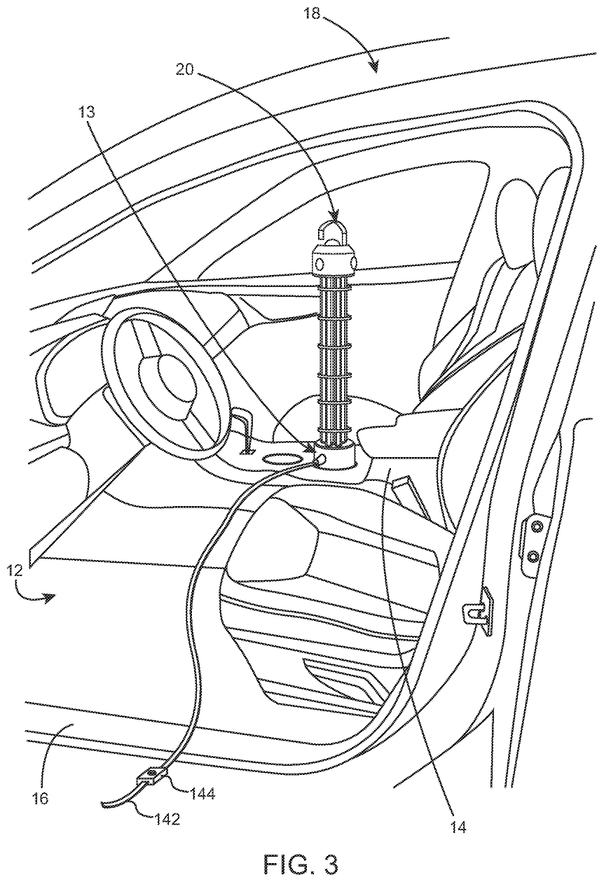

Referring to FIG. 3, an exemplary sanitizing UV lamp device 20 is shown supported by cup base housing 80 received within a cup holder 13 of a center console 14 of a vehicle cabin 12. Advantageously, the power supply cord 144 can be a particularly flatly shaped cord that can transit across a vehicle door jamb 16 and fit between a door of doorway 18 and the jamb 16 when the door is closed, thereby protecting users from UV irradiation upon activation of the device 20 for sanitizing the surfaces within vehicle cabin 12. Advantageously, the safety switch 144 can be located in series along the length of the power supply cord 142 and the switch 144 located outside of the doorway 18 so that it remains accessible from outside the vehicle after the vehicle door of doorway 18 is closed and germicidal irradiation by UV lamps 40 is initiated.

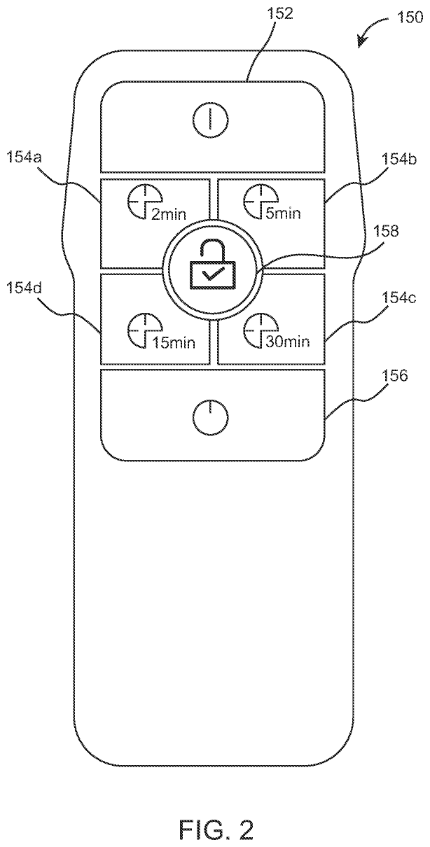

To facilitate activation of the device 20 by a user located outside of the area to be sanitized, for example outside of the vehicle cabin 12, the device 20 can include a wireless remote 150, for example as shown in FIG. 2. For example, the exemplary wireless remote 150 can include preset timer switches 154, for example, 2-minute timer switch 154a, a 5-minute timer switch 154b, a 15-minute timer switch 154d, and a 30-minute timer switch 154c. Additionally, the remote control 150 can include a power on switch 156, a power off switch 152, and a safety unlock switch 158. Unlock switch 158 provides a safety feature to prevent accidental activation of the device 20. More specifically, the unlock switch 158 must be pressed immediately preceding and within a preselected short duration of time within subsequent pressing of a switch of remote 150 used to activate the device 20, a timer switch 154 and power on switch 156. Only the power off switch 152 is active without pressing the unlock switch 158 in close time proximity to a subsequent switch actuation.

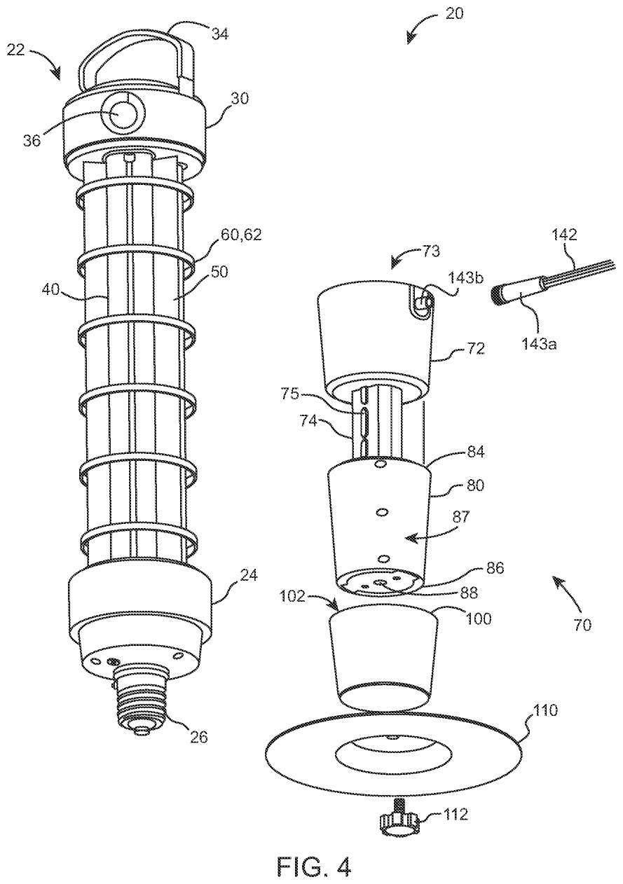

Referring to FIG. 4, the upper lamp support portion 22 may be separable from the lower base support portion 70, and the lower base support portion 70 may include a height adjustable feature. To facilitate separation of lamp support portion 22 from base support portion 70, lower housing 24 of lamp support portion 22 may include an electrical and mechanical connector that releasably couples with a corresponding connector of the lower base support portion 70. For example, the lower housing 24 may include a lamp base 26, for example, an industry standard E39 lamp base, for mechanic and electrical connection with a mating lamp base receptacle 73 located within a coupling housing 72 of the lower base support portion 70.

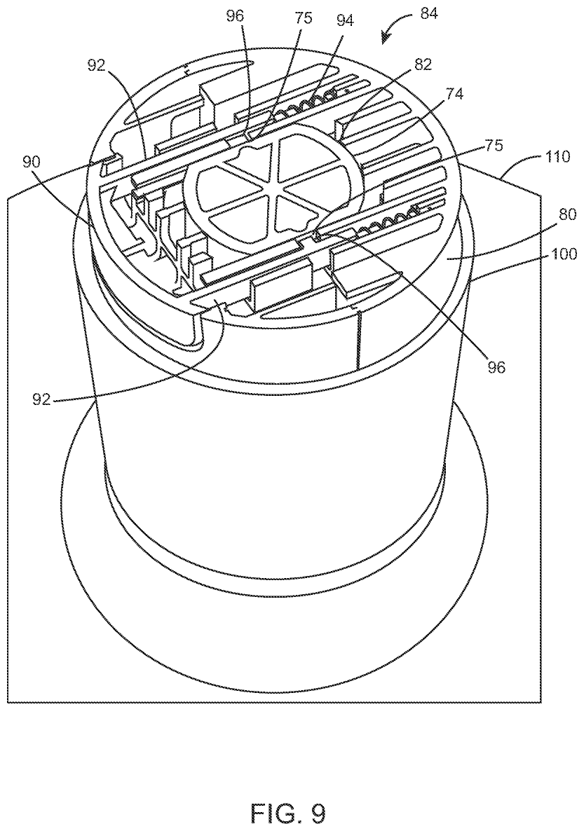

Lower base support portion 70 may also include a cup base housing 80 for supporting the device 20 within a range of sizes of industry standard cup holders. The lower base support portion also may include a height adjustment feature that provides for adjustment of the vertical distance between a bottom 86 of the cup base 80 and the UV lamps 40. For example, in the illustrative embodiment, a member, for example a height adjustment rod 74, extends from the coupling housing 72 and is adjustably and slidingly received into a cavity defined at the top end 84 of the cup base housing 80. For example, a cavity sized to slidingly receive the rod 74 may be defined by rod guides 82 at top end 84 of cup base housing 80 as illustrated in the cross-sectional view of FIG. 9.

The cup base housing 80 defines an outer surface 86 that is shaped and sized to be received within an industry standard sized cup holder 13, for example, as is typically found in a center consoles 14 of motor vehicle cabins 12. Advantageously, an optional cup base expander sleeve 100 defines a sleeve cavity 102 which is shaped to receive and support the cup base housing 80 and to provide an outer surface diameter to accommodate supporting the device 20 within a cup holder 13 having a larger size than that of cup base housing 80. Optionally, additional cup base expander sleeves having different sized outer surface diameters may also be included with device 20 as accessories. The cup base expander sleeves 100 may be formed from silicone rubber, or another flexible, semi-flexible, or rigid material.

Another accessory that may be included with the device 20 is a rigid, detachable base plate 110 that can be coupled to the bottom end 86 of the cup base housing 80, for example, using a thumbscrew 112 received by a threaded bore 88. The base plate 110 is sufficient diameter to steadily support the device 20 when resting up on a horizontal surface, for example, in an interior surface other than a motor vehicle cabin 12.

As illustrated in FIG. 5, the lower base support portion 70 includes a rotational lock actuator 76 for releasing the lamps support portion 22 for separation from the lower base support portion 70, and also includes a height adjust actuator 90 for enabling a height adjustment feature, for example, adjustment of the distance between the cup base housing 80 and the coupling housing 72, thereby changing the height of UV lamps 40 above the cup base housing 80.

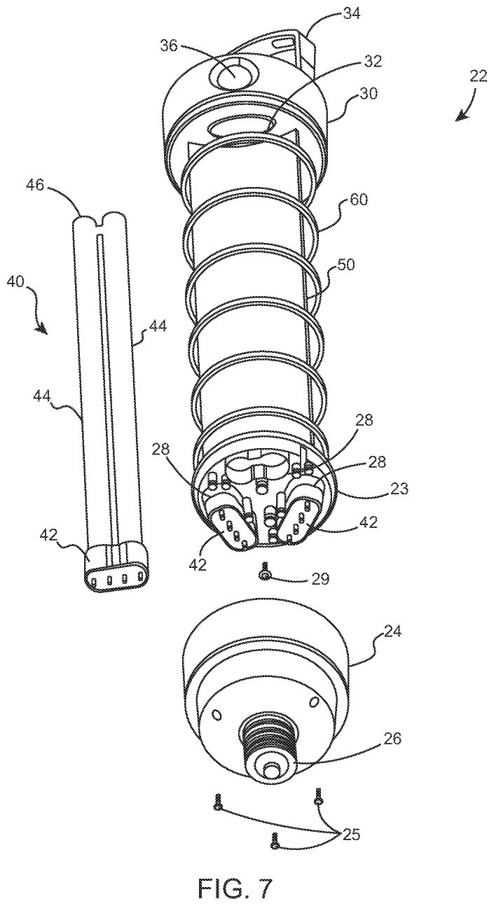

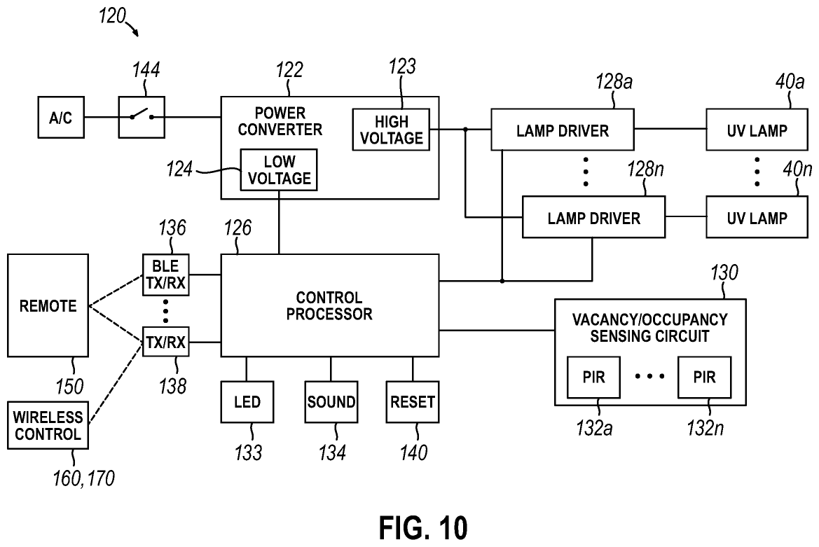

Short-wavelength UV lamps 40 that are effective for germicidal irradiation and that are readily commercially available, for example 254 nm, do degrade as time in use accumulates and therefore have a time-limited useful lifespan. Advantageously, the device 20 includes features to indicate elapsed use and/or remaining useful lifespan and to enable relatively easy replacement of the UV lamps 40. For example, the control process 126 (FIG. 10) can track expended and/or remaining lifespan and the LED indicator 133 may provide a blinking yellow or other notification that the UV lamps 40 need replaced. To facilitate easy replacement of the lamps 40, as shown in FIG. 7, the lamp support portion 22 can be separated from the lower base support portion 70, and the lower housing 24 can be separated from a lower housing cap 23 by removing fasteners 25.

Connector 42 of UV lamp 40 is located at an end of the lamp tubes 44 and is exposed upon separation of lower housing 24 from lower housing cap 23. Removal of the lower housing 24 may disconnect electrical connections providing power to the connector 42, or the connections maybe manually removed from the connector 42 to fully disconnect the lower housing 24 from the remaining lamp support portion 22. After removal of the lower housing 24, fastener 29 that retains connector 42 to lower housing cap 23 can be removed to enable removal of UV lamp 40 from lamp support portion 22. For example, lamp 40 can be slidably removed from within the bulb connector guide 28 defined by cap portion 23 of lower housing 24, withdrawing distal bulb end 46 from within a pocket or other recess define in upper housing 30, for example, lamp bulb receptacle 32 used to secure the distal bulb in 46 within the upper housing 30.

One the UV lamp 40 is fully removed from lamp support portion 22, reversing these steps can then be used to install a new UV lamp 40 within the bulb connector guide 28 and lamp bulb receptacle 32. All UV lamps 40 may be removed and replaced with a new lamp in this way, whether upon failure of the lamp or expiration of the useful time-limited lifespan.

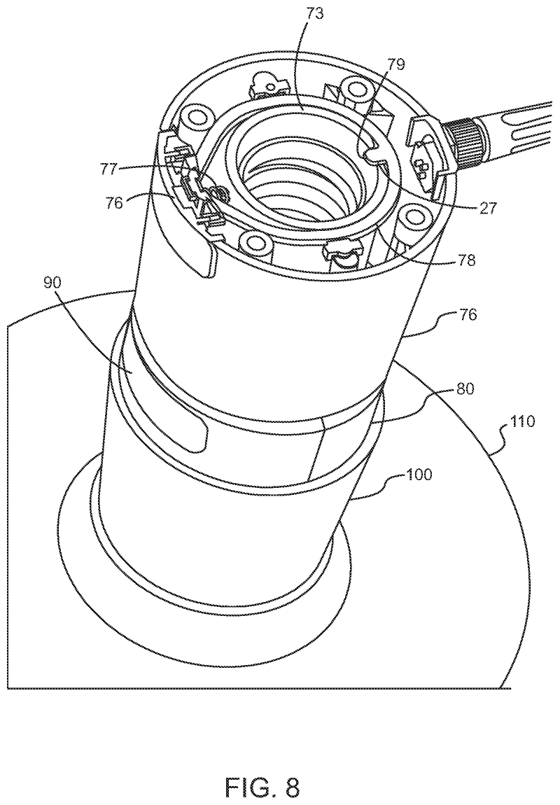

Referring to FIG. 8, rotational lock actuator 76 may be manually actuated to mechanically and electrically disconnect the lamp support portion 22 from the lower base support portion 70, including for example, to enable separate use of the lamp support portion 22 within a different powered lamp socket, or, for replacement of the UV lamps 40 as is discussed above.

More particularly, pressing the rotational lock actuator 76 against compression spring 77 translates locking ring 78 and locking tab 79 defined by the locking ring. The locking tab 79 is biased by the spring 77 into engagement with the upper portion lamp base 26 (FIG. 4), thereby preventing rotation and separation of the lamp base 26 from engagement within the lamp base receptacle 73. Upon actuation of the rotational lock actuator 76, tab 79 is translated away from the lamp base 26 and lamp base receptacle 73, thereby freeing the lower housing and the remainder portions of lamps support portion 22 to be rotated relative to the coupling housing 72 for electrical and mechanical separation of base 26, receptacle 73 and the associated housings. Upon mechanical reengagement of the lamp base 26 with the lamp base receptacle 73, and rotating into complete engagement, tab 79 is biased by spring 77 into engagement of the lamp base 26 rotational lock feature 27, thereby locking the lamp support portion 22 to the lower base support portion 70 until the rotational lock actuator is again actuated, for example, tab 79 biased into a vertically oriented recessed channel defined in lamp base 26. Other locking features known in the art may be substituted.

FIG. 9 illustrates locking and unlocking features for adjusting the height of the lamps 40 above the cup base 80 by adjusting the distance between coupling housing 72 and cup base 80. Rod 74 is attached to the coupling housing 72 and is slidably received within a receiving cavity defined by various rod guides 82 defined within the top end 84 of the cup base housing 80. As illustrated in FIG. 4, rod 74 may include locking features 75, for example segmented protrusions defined by a wall of the rod 74 and forming gaps therebetween. Referring again to FIG. 9, locking tabs 96 are biased by compression springs 94 to locate the tabs within the gaps between adjacent locking features 75, thereby preventing vertical sliding movement of the rod 74 within the guides 82 because of interference between the tabs 96 and the protrusion locking features 75.

Upon pressing the height adjust actuator 90, the tines 92 supporting tabs 96 on each side of the rod 74 are withdrawn from the gap between locking feature 75, thereby freeing the rod 74 to be slidingly translated up or down within the guides 82, thereby adjusting the distance between the top end 84 of cup base 80 and coupling housing 72. Upon release of the height adjust actuator 90, springs 94 bias the tines 92 and associated tabs 96 to again be positioned within the gaps between adjacent locking features 75, thereby locking the rod 74 relative to the guides 82, and fixing the position of coupling housing 72 relative to the cup base 80. In the exemplary embodiment, the height is adjustable by at least about 2 inches. Other locking features known in the art may be substituted for fixing the height adjustment position.

Referring to FIG. 10, an exemplary electrical circuit 120 of the sanitizing UV lamp device 20 is illustrated in schematic block diagram form. Electrical power, for example typical 120 Volt AC, can be provided by the power supply cord 142, which includes a series connected safety shutoff switch 144, for example a normally closed switch. A power converter 122 can include power conversion and/or power conditioning circuits, for example, including a low voltage supply 124 and a high voltage supply 123. The high voltage supply 123 provides power to lamp drivers 128a-128n, for example, individual resonant or other driver configurations each dedicated to an individual UV lamp 40a-40n. Separate drivers 128 enhance reliability and precise supply power for the UV lamps 40. The lamp drivers 128 are controlled by a control processor 126, and lamp drivers 128 may include a mechanical relay or solid-state switching device appropriate for connecting and disconnecting the high voltage supply 123.

Vacancy and/or occupancy sensing circuit 130 can include motion sensors, for example, PIR sensors 132a-132n, for detecting any motion proximate to the device 20. Because germicidal radiation from UV lamps 40 presents a risk to humans and animals, ensuring the vehicle cabin 12 or other interior area being sanitized is and remains vacant for the duration of an irradiation cycle is an important safety feature of the device 20.

The control processor 126 is powered by low voltage supply 124 and can include a digital controller and/or other electronic components powered by low voltage supply 124 for controlling the electrical operation of the device 20, including the illumination of UV lamps 40, interface with sensing circuit 130, circuit reset switch 140, LED indicator 133, sound emitter 134, wireless BLE transceiver 136, and other wireless transceivers 138, for example Wi-Fi, mesh radio, and/or another known wireless device standards.

Upon remote 150 being used to initiate a timed irradiation cycle of device 20, BLE transceiver 136 receives a signal from remote 150 and instructs the control processor 126 to begin a germicidal irradiation cycle. Control processor 126 and/or sensing circuit 130 ensures vacancy in the proximity of the device 20 through the entire cycle. For example, if occupancy is detected by sensing circuit 130 during a cycle, the cycle will disrupt power to UV lamps 40 to end the UV irradiation.

Upon initiation of a timed irradiation cycle and before control processor 126 instructs lamp drivers 128 to provide power to UV lamps 40 for the preselected germicidal irradiation time-period, the control processor 126 provides a delay warning period, for example 15-seconds, warning people to evacuate the area to be sanitized. For example, during the warning period, LED illuminator 133 can illuminate a specific color and/or sequence associated with the warning period, and sound emitter 134 can emit a warning sound, for example, a buzzer sound, alerting nearby personnel of the irradiation cycle that is about to begin, and providing the opportunity to evacuate from proximity of the device 20 or to stop the irradiation cycle before it starts.

Upon completion of the irradiation cycle, either by the expiration of the time delay countdown within control processor 126, or termination of the cycle by actuating the power off switch 152 of the remote 150, the control processor 126 will instruct lamp drivers 128 to terminate the power supplied to the UV lamps 40. Additionally, an added safety feature is that power can be interrupted via the safety switch 144 located along power supply cord 142, for example, which is located outside of the closed doorway 18 of the vehicle cabin 12 being sanitized.

Electrical circuit 120 of device 20 including control processor 126 may also be wirelessly operated by other wireless control devices in addition to or in place of wireless remote 150. For example, a smart device 160 including a specific application for operating device 20, or a Lightcloud enabled wireless controller 170, available from RAB Lighting Inc of Northvale, N.J., may be used with the control circuit 120. Such wireless controllers 160 and 170 can provide features similar to those provided by remote 150 and can advantageously provide additional features. For example, control processor 126 can capture use data, including elapsed UV lamp 40 time, and report this data to wireless controllers 160 and 170 and can be used for such advantages and warning when a useful lifespan for UV lamps 40 is approaching and/or is exceeded. Additionally, firmware updates for control processor 126 and other components of electrical circuit 120 maybe facilitated by wireless controllers 160 and 170. An indication of a firmware update needed or in progress can be provided by one or more of the LED indicator 133 and the sound emitter 134. Additionally, other optional features and parameters of device 20 as is known in the art for UV lamps used for germicidal irradiation can be viewed and/or set via wireless controller 160 and 170.

Advantageously, electrical circuit 120 of the device 20 can comprise a smart device, meaning, for example, the data processing and communication features of transceivers 136, 138 and control process 126 enable device 20 to be capable of controlling, cooperating, or being controlled by other IoT or other wireless devices, whether of the same type of device as device 20. Additionally, wireless control of one or more devices 20 can be extended by such wireless controllers 160 and 170 over a WAN for remote monitoring and control, for example, as disclosed by US Patent Application Publication 2021/0083897, published Mar. 18, 2021, and titled Wireless Lighting Controller for a Lighting Control System, hereby incorporated herein by references.

Advantageously, wireless remote 150 may be pre-paired with a device 20 for ease of use by an end user. Additionally, wireless remote 150 may be paired with one or more devices 20 using the specific application for operating devices 20 on the smart device 160. Advantageously, LED indicator 133 may illuminate a particular color and/or sequence to indicate a connection status with the wireless remote 150, for example, green if paired, and red or no indication if unpaired. Also, a wireless transceiver 138 configured for use with a mesh networks with wireless controller 170 may network devices 20 together and may be operated individually, or in one or more groups or sub-groups. Such operations individual or in one or more groups or sub-groups of devices 20 may also be implemented with BLE transceiver 136 or by transceiver 138 implementing another wireless radio standard known in the art. Advantageously, when devices 20 are selected to by operated as a group by wireless controller 150, 160, or 170, an added safety feature is that the whole group of devices 20 can be also configured to terminate an irradiation cycle upon the sensor circuit 130 detecting occupancy for any one of the devices 20 in the group.

Referring to FIG. 6, a reset switch 140, for example a recessed mechanical switch actuator that may be operated with a pointed object, can be located at a top surface of the upper housing 30 and can provide an electrical reset of a subset or all components of electrical circuit 120. Additionally, the LED indicator 133 may be located under and may illuminate the sensor cover 36 that is located at a top surface of the upper housing 30.

The control processor 126 and/or sensor circuit 130 can include features for detecting a failure of one of the UV lamps 40, the lamp drivers 128, or the occupancy and vacancy sensing circuit 130, including the PIR sensors 134, as is known in the art. For example, fault or failure detection may use an algorithm for detecting and comparing to expected data performance parameters such as voltage, current, and other electrical signal characteristics and/or data that can deviate from normal operation for the components monitored up a failure or other fault. Detection of such a defect can result in control processor 126 preventing operation of the device 20, and notification can be provided wirelessly to wireless controller 160 and 170. For example, in at least on embodiment, the control processor 126 queries the sensor circuit 130 every 20 ms. If the sensor circuit 130 files to respond to a query from the control processor 126, then the control processor 126 flags a fault and terminates the irradiation cycle, shutting of the UV lamps 40.

In an alternative embodiment, electrical circuit 120 may be powered by 12-volt DC, for example, as is available in most motor vehicle cabins 12. In at least one embodiment of the device 20, the coupling housing may include an electrical connector 143b for releasably coupling an electrical connector 143a of the power supply cord 142.

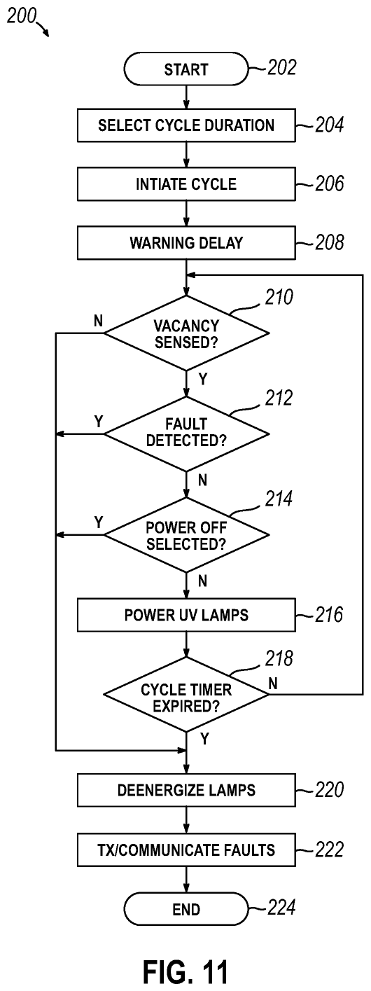

Referring to FIG. 11, an exemplary control algorithm 200 is illustrated. The control algorithm 200 can be executed by the components of the electrical circuit 120, including, for example, control processor 126, sensing circuit 130, and at least one of wireless remote controller 150, 160, and 170. At step 202, the control algorithm 200 begins.

At step 204, a user selects a cycle time duration using a wireless remote controller 150, 160, and 170, which then transmits this selection wirelessly to the control processor 126 via transceiver 136 or 138.

At step 206, control processor 126 initiates a germicidal irradiation cycle. At step 208, the control processor 126 provides a warning delay, for example, 15-second activation of one or more indicator, for example, LED indicator 133 and sound emitter 134, providing a warning of an cycle beginning shortly. After expiration of the warning delay at step 208, at step 210, one or more of the sensing circuit 130 and control processor 126 determines whether the space around the device 20 is vacant or occupied. If vacant, the algorithm continues to step 212. If occupied, the algorithm continues at step 220.

At step 212, one or more of the control processor 126 and the sensing circuit 130 determines whether a fault is detected, for example, absence of a response to a query sent from control processor 126 to the sensor circuit 130. If a fault is not detected, the algorithm continues at step 214. If a fault is detected, the algorithm continues at step 220.

At step 214, the control processor 126 receives and transmits any data communications pertaining to the current cycle. For example, receiving a data communication indicating the power off selection has been actuated on the wireless remote 150. If a power off selection is received, the algorithm continues at step 220. If not, the algorithm continues at step 216.

At step 216, the control processor 126 provides a control signal to the lamp drivers 128 instructing powering of the UV lamps 40. At step 218, the control processor 126 determines whether the preset time duration for the irradiation cycle has been completed. If not, the algorithm continues at step 210. If completed, the algorithm continues at step 220, and the control processor 126 deenergizes the lamps 40 via a control signal to the lamp drivers 128.

At step 222, the control processor 126 transmits via transceiver 136 or 138 and/or one or more of the LED indicator 133 and sound emitter 134 data identifying any detected fault and any other pertinent data regarding the irradiation cycle. At step 224, the algorithm is complete.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

Reference systems that may be used herein can refer generally to various directions (e.g., upper, lower, forward and rearward), which are merely offered to assist the reader in understanding the various embodiments of the disclosure and are not to be interpreted as limiting.

While examples, one or more representative embodiments and specific forms of the disclosure have been illustrated and described in detail in the drawings and foregoing description, the same is to be considered as illustrative and not restrictive or limiting. The description of particular features in one embodiment does not imply that those particular features are necessarily limited to that one embodiment. Some or all of the features of one embodiment can be used in combination with some or all of the features of other embodiments as would be understood by one of ordinary skill in the art, whether or not explicitly described as such. One or more exemplary embodiments have been shown and described, and all changes and modifications that come within the spirit of the disclosure are desired to be protected.

### Element Numbering

The following is a list of element numbers and at least one noun used to describe that element. The embodiments disclosed herein are not limited to these descriptions, and these element numbers can further include other words that would be understood by a person of ordinary skill reading and reviewing this disclosure in its entirety, and other words that may be used in other locations of this document.

| | | | |
|---|---|---|---|
| 12 | vehicle cabin | 32 | lamp bulb receptacle |
| 13 | cup holder | 34 | handle |
| 14 | center console | 36 | sensor cover |
| 16 | door jamb | 38 | reset switch |
| 18 | doorway | 40 | UV lamp |
| 20 | sanitizing UV lamp device | 42 | connector |
| 22 | lamp support portion | 44 | lamp tubes |
| 23 | cap portion | 46 | distal bulb end |
| 24 | lower housing | 50 | reflector |
| 25 | fasteners | 51 | wall |
| 26 | upper portion lamp base | 52 | apex |
| 27 | rotational lock feature | 60 | lamp screen |
| 28 | bulb connector guide | 62 | cage ring |
| 29 | lamp fastener | 70 | lower base support portion |
| 30 | upper housing | 72 | coupling housing |
| 73 | lamp base receptacle | 123 | high voltage supply |
| 74 | height adjust rod | 124 | low voltage supply |
| 75 | rod locking feature | 126 | lamp control processor |
| 76 | rotational lock actuator | 128 | lamp driver |
| 77 | spring | 130 | sensor circuit |
| 78 | locking ring | 132 | PIR sensors |
| 79 | locking tab | 133 | LED indicator |
| 80 | cup base housing | 134 | speaker |
| 82 | rod guides | 136 | BLE transceiver |
| 84 | top end | 138 | wireless transceiver |
| 86 | bottom end | 140 | reset switch |
| 87 | outer surface | 142 | power supply cord |
| 88 | threaded bore | 143 | connector |
| 90 | height adjust actuator | 144 | safety switch |
| 92 | tine | 150 | remote control |
| 94 | spring | 152 | power off switch |
| 96 | tab | 154 | timer switches |
| 100 | cup expander sleeve | 156 | power on switch |
| 102 | sleeve cavity | 158 | unlock switch |
| 110 | base plate | 160 | smart device |
| 112 | fastener | 170 | lighting controller |
| 120 | electrical circuit | 200 | control algorithm |
| 122 | power converter | | |

What is claimed is:

1. A sanitizing UV lamp device, comprising:
a housing including a cup shaped base;
a control circuit coupled to the housing;
at least one UV lamp coupled to the housing and selectively powered by the control circuit, the UV lamp providing a germicidal irradiation outside of the lamp housing;
a protective screen surrounding the at least one UV lamp; and
at least one motion detection sensor coupled to the control circuit; and
wherein:
the at least one UV lamp provides 360-degree germicidal irradiation through openings defined by the screen; and
the control circuit is configured to prevent irradiation from the at least one UV lamp upon detection of motion.

2. The sanitizing UV lamp device of claim 1, further comprising a removable sleeve sized to selectively overlay at least a portion of the cup shaped base, the removeable sleeve enabling the device to be supported at its cup shaped base by a range of motor vehicle cup holders.

3. The sanitizing UV lamp device of claim 1, further comprising a removable plate coupled to the cup shaped base, the removable plate enabling the device to be supported securely upon a horizontally oriented surface.

4. The sanitizing UV lamp device of claim 1, further comprising a height adjustment member providing an adjustable distance between the at least one UV lamp and the cup shaped base.

5. The sanitizing UV lamp device of claim 2, further comprising a coupling housing coupled to the at least one UV lamp, and wherein:
the height adjustment member includes a rod slidably received by guides for selectively displacing the coupling housing relative to the cup shaped based.

6. The sanitizing UV lamp device of claim 5, further comprising an actuator for releasing the rod to translate within the guides, thereby adjusting the distance between the at least one UV lamp and the cup shaped base.

7. The sanitizing UV lamp device of claim 1, wherein the control circuit is configured to detect motion sensor faults and disable irradiation of the at least one UV lamp upon fault detection.

8. The sanitizing UV lamp device of claim 1, wherein the control circuit further comprises at least one of a visible light emitter and an audible sound emitter, and upon initiating irradiation by the at least one UV lamps, during a delay period of time prior to irradiating the at least one of a visible light emitter and an audible sound emitter are activated by the control circuit to provide warning of irradiation initiating.

9. The sanitizing UV lamp device of claim 1, wherein:
the housing includes a lamp support portion supporting the at least one UV lamp between an upper housing and a lower housing;
at least a portion of one of the upper and the lower housing is selectively detachable to allow the at least one UV lamp to be removed and replaced by a new UV lamp.

10. The sanitizing UV lamp device of claim 9, wherein one of the upper and the lower housing includes a lamp receptacle for receiving a first end of the at least one UV lamp, and the other of the upper and the lower housing includes a lamp guide for slidably receiving a second end of the at least one UV lamp.

11. The sanitizing UV lamp device of claim 1, further comprising a reflector, and wherein the housing includes a lamp support portion supporting the reflector and supporting the at least one UV lamp between an upper housing and a lower housing.

12. The sanitizing UV lamp device of claim 11, wherein the housing includes a coupling device for selectively electrically and mechanically coupling the lamp support portion to the cup shaped base.

13. The sanitizing UV lamp device of claim 12, wherein the coupling device includes a screw type lamp base and lamp base receptacle.

14. The sanitizing UV lamp device of claim 11, further comprising a carrying handle coupled to the upper housing.

15. The sanitizing UV lamp device of claim 11, wherein:
the at least one UV lamp comprises at least three lamps;
the reflector comprises at least three reflecting walls forming a closed structure; and
each of the at least one UV lamps is supported adjacent each of the at least three reflecting walls.

16. The sanitizing UV lamp device of claim 1, further comprising a wireless remote controller, and wherein:
the control circuit includes a wireless transceiver in communication with the wireless remote controller; and
the wireless remote controller and the control circuit are configured to enable initiation of irradiation of the at least one UV lamp for a selected period of time.

17. The sanitizing UV lamp device of claim 16, wherein the wireless remote controller includes a plurality of preset period of time selectors and a safety unlock selector which must be pressed in sequence before selecting a switch to initiate irradiation.

18. The sanitizing UV lamp device of claim 16, wherein the wireless remote controller is an application operating on a handheld smart device.

19. The sanitizing UV lamp device of claim 16, wherein the wireless remote controller includes a wireless lighting control system capable of also wirelessly controlling room illumination lighting fixtures.

20. A sanitizing UV lamp device, comprising:
a housing including a cup shaped base;
a control circuit coupled to the housing; and
at least one UV lamp coupled to the housing and selectively powered by the control circuit, the UV lamp providing a germicidal irradiation outside of the lamp housing.

21. The sanitizing UV lamp device of claim 20, further comprising a protective screen surrounding the at least one UV lamp, and wherein the at least one UV lamp provides 360-degree germicidal irradiation through openings defined by the screen.

22. The sanitizing UV lamp device of claim 21, where the control circuit includes at least one motion detection sensor and the control circuit configured to prevent irradiation from the at least one UV lamp upon detection of motion.

23. The sanitizing UV lamp device of claim 21, further comprising a height adjustment member providing an adjustable distance between the at least one UV lamp and the cup shaped base.

24. The sanitizing UV lamp device of claim 21, further comprising a removable sleeve sized to selectively overlay at least a portion of the cup shaped base, the removeable sleeve enabling the device to be supported at its cup shaped base by a range of motor vehicle cup holders.

25. The sanitizing UV lamp device of claim 21, further comprising a protective screen surrounding the at least one UV lamp.

26. The sanitizing UV lamp device of claim 21, further comprising a plurality of motion detection sensors coupled to the control circuit.

27. The sanitizing UV lamp device of claim 21, further comprising a wireless remote controller, and wherein:
the control circuit includes a wireless transceiver in communication with the wireless remote controller; and
the wireless remote controller and the control circuit are configured to enable initiation irradiation of the at least one UV lamp for a selected period of time.

28. A sanitizing UV lamp device, comprising:
a housing
a control circuit coupled to the housing;
at least one UV lamp coupled to the housing and selectively powered by the control circuit, the UV lamp providing a germicidal irradiation outside of the lamp housing; and
a protective screen surrounding the at least one UV lamp, and wherein the at least one UV lamp provides 360-degree germicidal irradiation through openings defined by the screen; and
wherein the control circuit includes at least one motion detection sensor and the control circuit configured to prevent irradiation from the at least one UV lamp upon detection of motion.

29. The sanitizing UV lamp device of claim 28, wherein the housing includes a cup shaped base.

30. The sanitizing UV lamp device of claim 28, further comprising a height adjustment member providing an adjustable distance between the at least one UV lamp and the cup shaped base.

31. The sanitizing UV lamp device of claim 28, further comprising a wireless remote controller, and wherein:
the control circuit includes a wireless transceiver in communication with the wireless remote controller; and
the wireless remote controller and the control circuit are configured to enable initiation irradiation of the at least one UV lamp for a selected period of time.
